(19) United States
(12) Patent Application Publication
(10) Pub. No.: US 2012/0202275 A1
(43) Pub. Date: Aug. 9, 2012

(54) SYSTEM AND METHOD FOR REMOVING AIRBORNE CONTAMINANTS FROM EXHAUST GAS

(76) Inventor: Luis Uribe, Scottsdale, AZ (US)

(21) Appl. No.: 13/365,068

(22) Filed: Feb. 2, 2012

Related U.S. Application Data
(60) Provisional application No. 61/439,424, filed on Feb. 4, 2011.

(51) Int. Cl. C12S 5/00; C12M 1/12

(52) U.S. Cl. 435/266; 435/297.1

(57) ABSTRACT

The present invention provides a system and method for purifying and/or filtering gases. The system and method remove undesired species from a gas through the use of bioorganisms which reside in and on a filtration media. Water and nutrients can be supplied to the filtration media to promote the growth and biological activity of the bioorganisms.

### CROSS-REFERENCE TO RELATED APPLICATIONS

This application claims priority to U.S. Provisional Application Ser. No. 61/439,424, entitled “SYSTEMS AND METHODS FOR REMOVING AIRBORNE CONTAMINANTS FROM EXHAUST GAS” filed Feb. 4, 2011, which is hereby incorporated by reference in its entirety.

### FIELD OF INVENTION

The invention relates to systems for removing airborne contaminants from exhaust gas. In particular, the invention relates to systems for removing airborne contaminants and other undesired species, such as hydrogen sulfide and other sulfide compounds, from exhaust gases through the use of microorganisms.

### BACKGROUND OF THE INVENTION

Wastewater produces foul air, which is dangerous, corrosive and regulated. Current systems that attempt to remove and/or eliminate hydrogen sulfide and other airborne contaminants have limitations, including difficulties in maintaining and/or replacing filtration media and poor bioorganism adherence to filtration media.

An exemplary gas filtration and purification system of the present disclosure provides for simpler and less expensive filtration media replacement. In addition, bioorganism adherence in the filtration media can be improved. Finally, the filtration of undesired species from waste gas can be improved by improving the airflow distribution throughout the filtration media.

### SUMMARY OF THE INVENTION

In accordance with exemplary embodiments, a system and method for purifying gases by removing undesired contaminants, such as hydrogen sulfide and other sulfide compounds, from exhaust gases through the use of microorganisms is provided. In accordance with an exemplary embodiment, a system for purifying and/or filtering contaminants from gas comprises a filtration chamber with at least one filtration stage. In various exemplary embodiments, the filtration chamber is substantially rectangular and/or square.

In various exemplary embodiments, the filtration stage comprises a filtration media which includes at least one layer of structural filtration material and at least one layer of bioorganism fostering filtration material. The structural filtration material is configured to support the at least one layer of bioorganism fostering filtration material. In various exemplary embodiments, the filtration media is substantially rectangular. In various exemplary embodiments, at least one layer of each type of material can be stacked vertically or horizontally.

Various exemplary embodiments may further comprise a gas inlet, an exhaust gas outlet; and a water inlet, and/or a control section.

### BRIEF DESCRIPTION OF THE DRAWINGS

The exemplary embodiments of the present invention will be described in conjunction with the accompanying drawing figures in which like numerals denote like elements and:

FIG. 1 is a cross-sectional side view illustrating an exemplary system for purifying gas;

FIGS. 2A, 2B, 2C, 2D, and 2E are cross-sectional views of exemplary filtration media;

FIG. 3 is a cross-sectional top view illustrating an exemplary system for purifying gas; and

FIG. 4 is a top view illustrating an exemplary system for purifying gas.

### DETAILED DESCRIPTION

Persons skilled in the art will readily appreciate that various aspects of the present invention can be realized by any number of systems and methods configured to perform the intended functions. Stated differently, other systems and methods can be incorporated herein to perform the intended functions. It should also be noted that the accompanying drawing figures referred to herein are not all drawn to scale, but can be exaggerated to illustrate various aspects of the present invention, and in that regard, the drawing figures should not be construed as limiting. Finally, although the present invention can be described in connection with various principles and beliefs, the present invention should not be hound by theory.

In various exemplary embodiments, gas purification systems of the present disclosure comprise a filtration chamber which includes at least one filtration stage. The filtration stage or stages include a filtration media, and bioorganisms are cultivated within the filtration media itself. The filtration media comprises at least one layer of structural filtration material and at least one layer of bioorganism fostering filtration material. In various embodiments, the filtration media is substantially rectangular and/or square. Exemplary gas purification systems further include a gas inlet, water inlet, and exhaust gas outlet.

In various exemplary embodiments, incoming gas, such as waste gas, enters the filtration system, and the bioorganisms on and in the filtration media scrub the incoming gas by removing undesired species, such as hydrogen sulfide, through biological processes.

Water is added to the filtration system, typically at the top of the filtration chamber, and allowed to trickle down to the bottom, This water fosters the bioorganism growth, as well as washes away biological byproducts created by the bioorganisms. Exhaust gas can exit the filtration system after passing through the filtration stages.

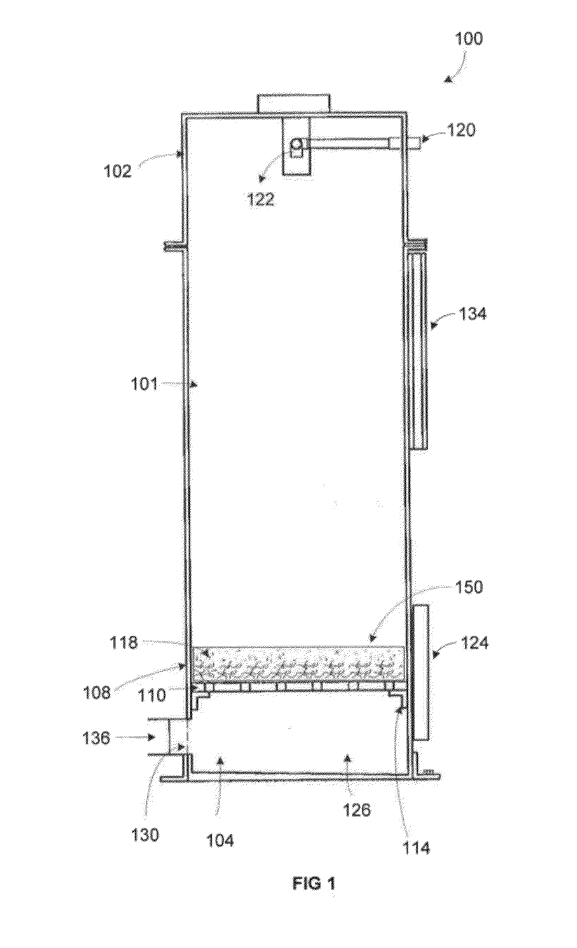

With initial reference to FIG. 1, an exemplary gas filtration system 100 is illustrated. Gas filtration system 100 comprises a filtration chamber 101. Filtration chamber 101 can comprise, for example, a vertical chamber which allows gas to flow through and be purified. In various exemplary embodiments, filtration chamber 101 comprises a square or rectangular shape. In contrast to a round filtration chamber, a square or rectangular filtration chamber represents a more efficient use of space, as the surface area or footprint of the chamber can be fully utilized. However, any size and shape of filtration chamber 101 is within the scope of the present invention.

In various exemplary embodiments, filtration chamber 101 comprises fiberglass, fiberglass reinforced plastic (FRP), or other corrosion resistant polymers. However, filtration chamber 101 can comprise any material that provides sufficient support for filtration stages and allows the filtration system to operate within the desired parameters.

In various exemplary embodiments, filtration chamber 101 comprises at least one filtration stage 108, For example, filtration stage 108 can comprise horizontal stages which are vertically stacked within filtration chamber 101. In various embodiments, a number of filtration stages 108 are equally spaced apart from each other. In other embodiments, a number of filtration stages 108 can be spaced apart from each other at varying distances,

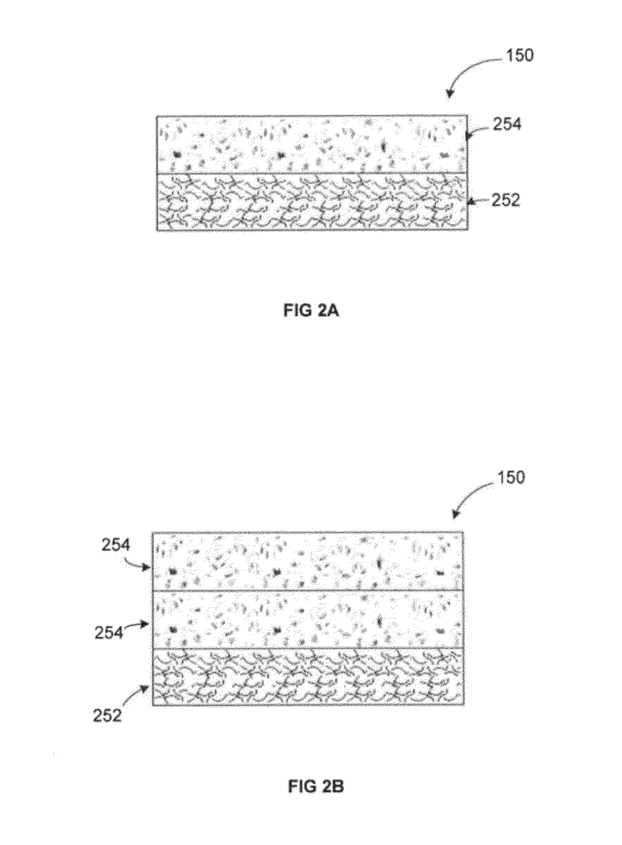

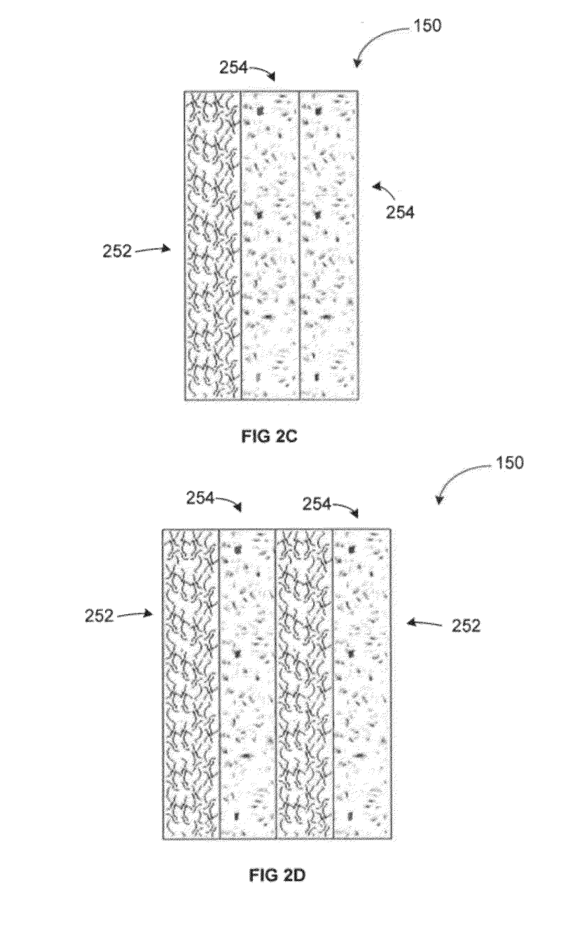

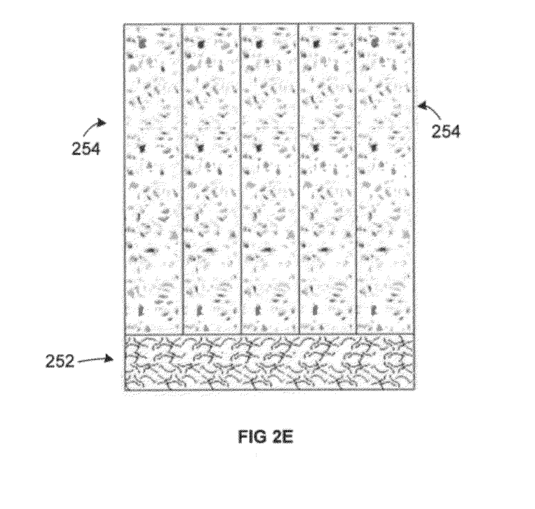

In various exemplary embodiments, filtration stage 108 comprises a filter media 150. In various embodiments, filtration media 150 provides a substrate for the growth and support of bioorganisms. Filtration media 150 can comprise a combination of multiple types of filtration material. For example, with initial reference to FIGS. 2A and 2B, filter media 150 can comprise one or more layers of structural filtration material 252 and bioorganism fostering filtration material 254.

In various exemplary embodiments, structural filtration material 252 can comprise material which provides rigidity, structure, and/or support to the other layers, and allows both gas and liquid to flow through the material. For example, structural filtration material 752 can comprise non-woven nylon, cellular foam, polystyrene, and/or polypropylene. In various embodiments, filtration media 150 comprises corrosion-resistant filtration material or materials.

In various exemplary embodiments, bioorganism fostering filtration material 254 can comprise a material capable of fostering and supporting the growth of suitable bioorganisms. Because bioorganism fostering filtration material 254 is used in combination with structural filtration material 252, bioorganism fostering filtration material 254 can comprise less rigid materials. For example, bioorganism fostering filtration material 254 can comprise a polymeric material, such as cellular foam, sponge foam, polystyrene and/or polypropylene. Any material capable of fostering bioorganism growth, and is capable of allowing liquid and gas to flow through the material, is within the scope of the present disclosure.

In various exemplary embodiments, filtration media 150 can comprise a stack of horizontally oriented layers of filtration material. For example, structural filtration material 252 and bioorganism fostering filtration material 254 can be cut into squares and stacked vertically. In an exemplary embodiment, layers of materials are alternated, such that the first layer is of one material, and the second layer is of another material. In another exemplary embodiment, a layer of structural filtration material 252 can be placed in the vertical stack with one or more different types of bioorganism fostering filtration material 254. In various embodiments, filtration media 150 comprises a plurality of cubes, each cube comprised of alternating layers of structural filtration material 252 and bioorganism fostering filtration material 254. However, any combination and configuration of structural filtration material 252 and bioorganism fostering filtration material 254 which provides an effective filtration media to remove undesired species from inlet gas is within the scope of the present disclosure.

In other exemplary embodiments, filtration media 150 can comprise layers of filtration material arranged vertically, For example, as illustrated in FIGS. 2C and 2D, a number of layers of structural filtration material 252 and bioorganism fostering filtration material 254 are oriented vertically, such that layers of structural filtration material 252 sandwich one or more layers of bioorganism fostering filtration material 254. As is the case with horizontally-oriented filter media 150, in a vertical orientation, structural filtration material 252 can provide rigidity, structure, and/or support to the bioorganism fostering filtration material 254.

In yet other exemplary embodiments, filtration media 150 can comprise layers of filtration media arranged vertically and horizontally. For example, with reference to FIG. 2E, multiple layers of bioorganism fostering filtration material 254 can be vertically arranged. One or more layers of structural filtration material 252 can be horizontally arranged such that it supports the vertical arranged layers of bioorganism fostering filtration material 252. However, as discussed above, any combination and configuration of materials 252 and 254 which provides an effective filtration media is in accordance with the present disclosure.

In an exemplary embodiment, filtration chamber 101 further comprises filtration bioorganisms 118, in various embodiments, the growth of filtration bioorganisms 118 is fostered and encouraged on the bioorganism fostering filtration material 254 of filtration media 150. As polluted air passes through filtration chamber 101, bioorganisms 118 can remove, through digestion or other biological process, pollutants from the polluted air. For example, hydrogen sulfide can be removed from polluted air by the biological activity of bioorganisms 118. In an exemplary embodiment, bioorganisms 118 from the genus Thiobacillus are grown and fostered in filtration media 150. However, any bioorganisms 118 which can remove undesired species from polluted air in the environment provided by filtration chamber are in within the scope of the present disclosure.

In various exemplary embodiments, the type of bioorganisms 118 can be determined by the incoming water. For example, bioorganisms present in the incoming water may attach to and grow on the bioorganism fostering filtration material 254 of filtration media 150. In various exemplary embodiments, incoming water can comprise a sludge which includes a wide variety of different types of bioorganisms.

In various exemplary embodiments, the type of bioorganisms 118 can also be determined by the composition of the incoming gas. Bioorganisms present on bioorganism fostering filtration material 254 can feed on components of the incoming gas. For example, if the incoming gas contains hydrogen sulfide gas, bioorganisms such as Thiobacillus present on bioorganism fostering filtration material 254 can feed on the hydrogen sulfide, fostering their growth.

In various exemplary embodiments, the types of bioorganisms 118 present in filtration media 150 may be impacted by aspects of filtration media 150. For example, changes in the number, relative heights, and types of materials of filtration media 150 can change the operating pH range of the filtration system. At different pH ranges, different bioorganisms 118 can be cultivated. Particular bioorganisms can be selected based on their ability to remove a particular undesired species from an inlet gas. Therefore, in various exemplary embodiments, the composition of filtration media 150 can be changed to cultivate the particular bioorganism or bioorganisms 118 that will be most effective in removing a specific undesired species.

In various embodiments, filtration stages 108 further comprise at least one support grate 110. Support grate 110 provides support for filtration media 150. For example, filtration media 150 can be arranged across the surface of a horizontal support grate 110. In this configuration, support grate 110 would bear the weight of filtration media 150, which can include a high liquid content and biological component. In an exemplary embodiment, support grate 110 comprises a corrosion-resistant material that strong enough to support the wetted filtration media 150. For example, support grate 110 can comprise fiberglass reinforced plastic or polymer-coated metals and the like. However, any material, whether or not corrosion-resistant, which provides adequate support for filtration media 150 and permits both air and liquid to pass through to the adjacent stages is within the scope of the present disclosure.

In various exemplary embodiments, filtration chamber 101 further comprises at least one support grate reinforcement member 114. In various embodiments, support grate reinforcement member 114 is a horizontal support which provides additional structural reinforcement to support grate 110. In other embodiments, support grate reinforcement members 114 can be vertical supports, such as columns. However, any member that provides additional structural reinforcement to support grates 110 and permits both air and liquid to pass through to the adjacent stages can be utilized.

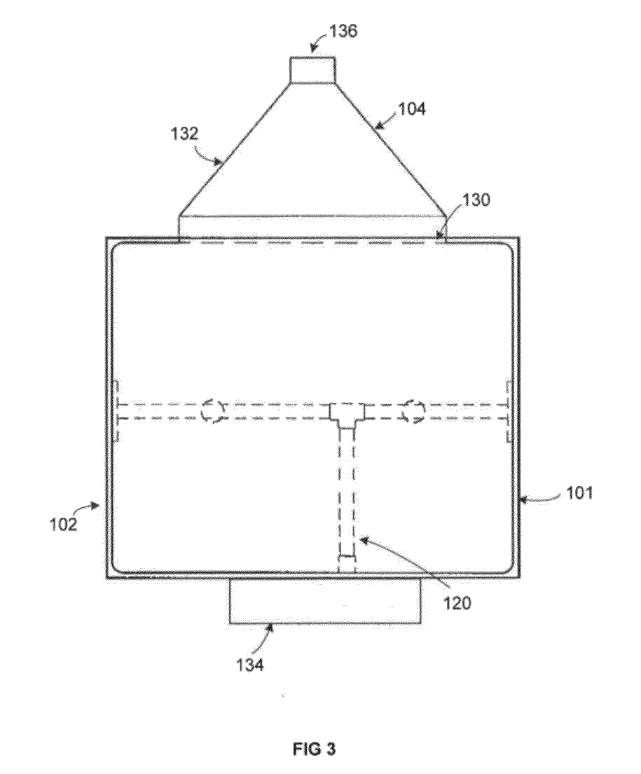

With reference to FIGS. 1 and 3, in various exemplary embodiments, gas filtration system 100 further comprises a water inlet 120. Water inlet 120 provides a flow of water to filtration media 150, which can be necessary for the growth of bioorganisms 118. In various embodiments, water provided to gas filtration system 100 by water inlet 120 can comprise potable water. In other embodiments, water provided to gas filtration system 100 by water inlet 120 can comprise secondary effluent, such as reclaimed or recycled water.

In an exemplary embodiment, water inlet 120 comprises at least one nozzle 122 which is situated at the top of filtration chamber. Nozzles 122 provide water at the top of filtration chamber 101, and the water flows downward through the filtration stages 108. In addition, other operating parameters, such as pH levels, can be controlled or adjusted by the number, spray angle, and position of nozzles 122 in the filtration chamber 101. For example, changing the distance between the nozzles 122 and the closest filtration stage 108 can affect the pH levels at various points in the filtration chamber 101.

With continued reference to FIG. 3, in various exemplary embodiments, gas filtration system 100 can comprise a nutrient source 124. In such embodiments, for example, nutrients from nutrient source 124 can be provided to water inlet 120, and delivered by at least one nozzle 122 to the top of filtration chamber 101. This provides nutrients to bioorganisms 118 of filtration stages 108. Nutrients can include any mineral or compound which aids in the growth or subsistence of bioorganisms 118.

In various embodiments, nutrient source 124 is a removable vessel. In such configurations, nutrient source 124 can he removed from gas filtration system 100 and replenished with sufficient nutrients. In other embodiments, nutrient source 124 can be a continuous supply of nutrients, such as a low pressure and/or drip system. However, any manner of providing nutrient source 124 to gas filtration system 100 is within the scope of the present disclosure.

In various exemplary embodiments, gas filtration system 100 further comprises a gas inlet 104. Gas inlet 104 can introduce fouled or polluted air into filtration chamber 101 to be scrubbed. In various exemplary embodiments, gas inlet 104 is configured at the bottom of the filtration chamber 101, such that the incoming gas rises up through filtration chamber 101. For example, with reference to FIGS. 1 and 3, gas inlet 104 can comprise a funnel-type structure, in which gas is pumped from a large tube or pipe 136, enters an enlarging duct 132, and is directed towards the bottom of filtration chamber 101.

With reference to FIG. 3, gas inlet 104 can further comprise a gas distribution member 130. For example, gas distribution member can comprise a perforated plate which is installed at the point where the gas enters the bottom of filtration chamber 101. In such configurations, gas distribution member 130 acts to disperse the incoming gas and create a more even flow distribution into filtration chamber 101.

In various exemplary embodiments, gas filtration system 100 further comprises a gas outlet 106. Gas outlet 106 provides an exit for gas that has been scrubbed or treated by gas filtration system 100. In an exemplary embodiment, gas outlet 106 comprises an exhaust pipe 140 located above the highest filtration stage 108. However, any manner of exhausting scrubbed or treated gas from the filtration system is in accordance with the present invention.

In various exemplary embodiments, and with reference to FIG. 1, gas filtration system 100 comprises a housing 102. Housing 102 can be configured to contain various components of gas filtration system 100, such as filtration chamber 101. In various embodiments, components such as gas inlet 104, gas outlet 106, water inlet 120, and/or nutrient source 124.

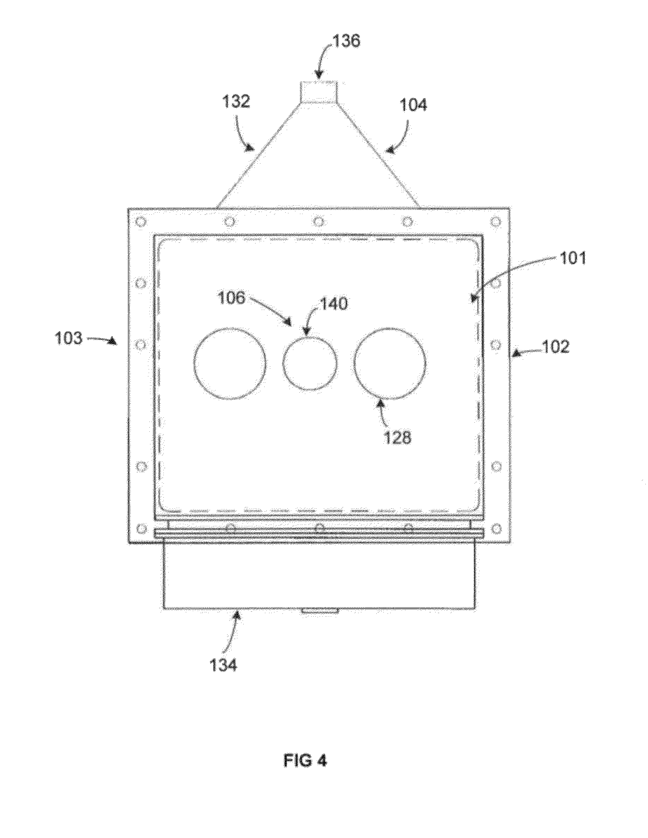

With reference to FIG. 4, in various exemplary embodiments, housing 102 further comprises a cap section 103. Cap section 103 can be attached to the top of the housing 102 by removable means, such as bolts or clamps. In an exemplary embodiment, cap section 103 includes at least one inspection port 128. Inspection port 128 allows an operator to inspect the condition and operation of the system without removing the entire cap section 103.

With reference to FIG. 1, in various exemplary embodiments, gas filtration system 100 can further comprise a drain section 126. Drain section 126 can be configured at the bottom of the filtration chamber 101. In an exemplary embodiment, drain section 126 is below the lowest filtration stage 108. Drain section 126 allows the water which has travelled through the various filtration stages to exit filtration chamber 101. Typically, this waste water includes byproducts created by bioorganisms 118, such as salts and/or acids. As a result, it is often acidic, with a pH as low as 2. In an exemplary embodiment, drain section 126 can comprise a sump which allows for collection of any waste water prior to disposal and facilitates the safe collection, treatment, and disposal of waste water. In various exemplary embodiments, drain section 126 can comprise a sloped floor which directs water to a drain. In an exemplary embodiment, drain 126 can comprise both a sloped floor and a sump.

In an exemplary embodiment, gas filtration system 100 further comprises a control section 134. Control section 134 can include controls to regulate the operation of gas filtration system 100. For example, control section 134 can include controls to regulate the inlet and exhaust gas flow rates, water supply pressure, nutrient distribution, temperature, pressure, and humidity within the filtration chamber. Control section 134 can receive data from various sensors within filtration chamber 101. Control section 134 can also include instruments to display operating parameters such as gas flow rates, water supply pressure and flow rate, humidity, temperature and pressure inside filtration chamber 101, and pH at various stages 108 within filtration chamber 101. Control section 134 can also be configured to transmit data and instrument readings to remote instruments and/or remote terminals.

In various exemplary embodiments, control section 134 comprises a manual control system. In an exemplary manual control section 134, an operator can utilize control valves to adjust operating parameters, such as flowrates, temperature, pressure, and humidity. In other exemplary embodiments, control section 134 comprises an automatic control system. In an exemplary automatic control section 134, operating parameters can be predetermined and set, and control section 134 can adjust conditions within filtration chamber 101 to achieve the desired parameters. For example, an automatic control section 134 can maintain a constant temperature, pressure, and/or humidity as the filtration system is operating. Any method of controlling the operating parameters of the filtration system is in accordance with the present disclosure.

The foregoing disclosure is merely illustrative of the present invention and is not intended to he construed as limiting the invention. Although one or more embodiments of the present invention have been described, persons skilled in the art will readily appreciate that numerous modifications could be made without departing from the spirit and scope of the present invention. As such, it should be understood that all such modifications are intended to he included within the scope of the present invention.

We claim:

1. A system for purifying gases, the system comprising:
a filtration chamber;
at least one filtration stage located within the filtration chamber, wherein the filtration stage comprises a filtration media,
wherein the filtration media comprises at least one layer of structural filtration material and at least one layer of bioorganism fostering filtration material, and
wherein the filtration media is substantially rectangular;
a gas inlet in fluid communication with the filtration chamber;
an exhaust gas outlet in fluid communication with the filtration chamber; and
a water inlet in fluid communication with the filtration chamber.

2. The system of claim 1, further comprising a housing.

3. The system of claim 1, further comprising a control system configured to control the operating conditions within the filtration chamber.

4. The system of claim 1 wherein the at least one layer of structural filtration material and the at least one layer of bioorganism fostering filtration material are stacked horizontally.

5. The system of claim 1 wherein the at least one layer of structural filtration material and the at least one layer of bioorganism fostering filtration material are stacked vertically,

6. The system of claim 1 wherein the at least one filtration stage further comprises a support grate.

7. The system of claim 1, wherein the at least one filtration stage further comprises a reinforcement member.

8. The system of claim 1, wherein the bioorganism fostering layer comprises one of the group of polystyrene, polypropylene, and cellular foam.

9. The system of claim 1, wherein the bioorganism fostering layer comprises a nonwoven synthetic material.

10. The system of claim 1, wherein the structural material layer comprises one of the group of polystyrene, polypropylene, and cellular foam.

11. The system of claim 1, wherein the structural material layer comprises a nonwoven synthetic material.

12. The system of claim 1 wherein the filtration media is substantially square,

13. The system of claim 1, further comprising a nutrient source.

14. The system of claim 13, wherein the nutrient source is a removable vessel in fluid communication with the filtration chamber.

15. The system of claim 1, further comprising at least one nozzle.

16. The system of claim 15, wherein the at least one nozzle is located within the filtration chamber and is configured to deliver water to the filtration chamber.

17. The system of claim 1, further comprising an air inlet distribution member.

18. A method fur purifying gas comprising:
encouraging the growth of bioorganisms on a filtration media, wherein the filtration media is substantially rectangular and comprises at least one layer of structural filtration material and at least one layer of bioorganism fostering filtration material;
supporting the weight of at least one layer of bioorganism fostering filtration material and bioorganisms by stacking the at least one layer of bioorganism fostering filtration material with the at least one layer of structural filtration material;
providing a water supply to the filtration media;
providing the gas to the filtration media, wherein the filtration media allows the gas to pass through it; and
removing purified exhaust gas.

19. The method of claim 18, further comprising the step of providing nutrients to the bioorganisms.

20. The method of claim 18, wherein the at least one layer of structural filtration material and the at least one layer of bioorganism fostering filtration material are stacked vertically.
